(19) United States
(12) Patent Application Publication
(10) Pub. No.: US 2006/0165635 A1
(43) Pub. Date: Jul. 27, 2006

(54) Personal care formulations containing keratin

(76) Inventors: Robert James Kelly, Christchurch (NZ); Alisa Dawn Roddick-Lanzilotta, Christchurch (NZ)

Correspondence Address: BACON & THOMAS, PLLC, 625 SLATERS LANE, FOURTH FLOOR, ALEXANDRIA, VA, 22314, US

(21) Appl. No.: 10/536,325

(22) PCT Filed: Nov. 28, 2003
(86) PCT No.: PCT/NZ03/00263

§ 371 Date: Nov. 7, 2005

(30) Foreign Application Priority Data
Nov. 28, 2002 (NZ) 522836
Mar. 12, 2003 (NZ) 524706

(51) Int. Cl. A61K 8/65; A61K 8/64

(52) U.S. Cl. 424/70.14

(57) ABSTRACT

A range of personal care products that include a keratin protein fraction. The fraction may be intact or hydrolysed. It is preferably S-sulfonated. The content of the fraction may range from 0.001% to 50%. In most formulations its content will be less than 1% although in certain products such as nail care products the content will be higher. A wide range of personal care products are described including shampoos, body gels and lotions, conditioners, creams and cosmetics generally.

### FIELD OF THE INVENTION

The invention relates to personal care formulations containing keratin and their use in cosmetics.

### BACKGROUND OF THE INVENTION

Proteins and their derivatives are used in a wide range of personal care formulations, including those intended for use on the hair, skin and nails. As a component of personal care formulations, proteins perform many functions, including conditioning, film forming, as a humectant and an emollient Most commonly used proteins are hydrolysed in order to impart sufficient solubility to facilitate inclusion in a formulation This is particularly the case with keratin proteins, which are inherently insoluble due to the crosslinks associated with the characteristically high degree of cysteine present in the protein. Numerous examples of the use of hydrolysed proteins, including keratins, in personal care formulations are known in the art.

WO9851265 discloses the use of hydrolysed proteins and their derivatives, particularly those with high sulfur content, in formulations to protect hair from the insults of environmental and chemical damage. The inventors in WO9851265 use a combination of hydrolysed proteins and a polyamino cationic agent in order to prepare the desired formulations.

U.S. Pat. No. 4,948,876 describes an S-sulphocysteine keratin peptide produced by enzymatic hydrolysis for use as an auxiliary in the dyeing of wool and hair. Enzymatic digestion is used by the authors to prepare low molecular weight peptides and achieve the desired solubility.

U.S. Pat. No. 4,895,722 discusses the use of a range of keratin decomposition products, including those obtained by chemical and enzymatic hydrolysis, for the preparation of cosmetic products.

Keratin fibres, such as human hair, wool and other animal fibres, consist of a complex mix of related proteins that are all part of the keratin family. These proteins can be grouped according to their structure and role within the fibre into the following groups:
the intermediate filament proteins (IFP), which are fibrous proteins found mostly in the fibre cortex;
high sulfur proteins (HSP), which are globular proteins found in the matrix of the fibre cortex, as well as in the cuticle.
high glycine-tyrosine proteins (HGTP), found mostly in the fibre cortex.

The ultrastructure of keratin fibres is well known in the art, and discussed in detail by R. C. Marshall, D. F. G. Orwin and J. M. Gillespie, Structure and Biochemistry of Mammalian Hard Keratin, Electron Microscopy Reviews, 4, 47, 1991. In the prior art described in which proteins are used as a cosmetic ingredient, the keratin utilized is hydrolysed as one material, with no attempt at fractionating the keratin source into its constituent components. As a result of protein hydrolysis, many of the desirable properties of the proteins are lost. Low molecular weight keratin peptides aggregate with a much lower degree of order to produce materials with much poorer physical properties than the high molecular weight keratins from which they are derived. In addition, irreversible conversion of cysteine as may occur with chemical methods of keratin decomposition, yields a peptide product that has lost the core functionality that that distinguishes it from other protein materials.

The need exists for personal care formulations which use intact keratins which maintain many of the desirable characteristics of the native keratins from which they are derived and possess a reactivity towards keratin substrates.

### OBJECT OF THE INVENTION

It is an object of the invention to provide a personal care formulation which uses a keratin protein or to at least provide the public with a useful choice.

### SUMMARY OF THE INVENTION

The invention provides a personal care formulation including a keratin protein fraction.

The keratin protein fraction may be intact.

The invention also provides a personal care formulation in which the keratin protein fraction is hydrolysed.

In particular, the invention provides a personal care formulation including a keratin protein fraction which is S-sulfonated.

The invention provides personal care formulations in which the keratin protein fraction is from the intermediate filament protein family.

The invention also provides a personal care formulation in which the keratin protein fraction is from the high sulfur protein family.

The cysteine content of the keratin protein may be about 4%.

The invention also provides a personal care formulation in which the keratin protein fraction is from the high glycine-tyrosine protein family.

Preferably the percentage of the intact S-sulfonated keratin protein fraction in the formulation is less than ten percent by weight.

More preferably the ratio is between 0.001 and 1% inclusive by weight. However the ratio may be from 0.001% to 50% of keratin protein fraction.

The invention also provides a personal care formation containing about 0.001% to 50% of a keratin protein fraction.

The ratio is preferably 0.001% to 10% and more preferably 0.001% to 1%.

The invention also provides an additive for a personal care formation comprising a keratin protein fraction.

The personal care formulations may include the following:
Conditioning shampoo;
Body/Facial cleanser/shampoo;
Hair conditioner;
Hair gel;
Hair mouse, setting lotion;
Hairspray,
Pre-perming solution;
Post-perming solution;
Moisturing cream;
Shower gel;
Foaming bath gel;
Mascara;
Nail polish
Liquid foundation,
Shaving cream; and
Lipstick.

However other personal care formulations are included within the invention.

The invention also provides a personal care formulation including an intact sulfonated keratin fraction wherein the ratio of keratin fraction is about 10% of the formulation. The formulation is adapted to be used as a nail polish or nail glosser.

The personal care formulations comprise a suitable percentage by weight of a cosmetic carrier.

Additional elements such as vitamins and minerals may be added to enhance the protective efficacy of the formulations.

Sunscreen factors with ultra-violet protection properties may also be added.

The invention also provides a method of using the personal care formulation or additives according to the invention.

### DETAILED DESCRIPTION OF THE DRAWINGS

The invention will now be described by way of example only in which:

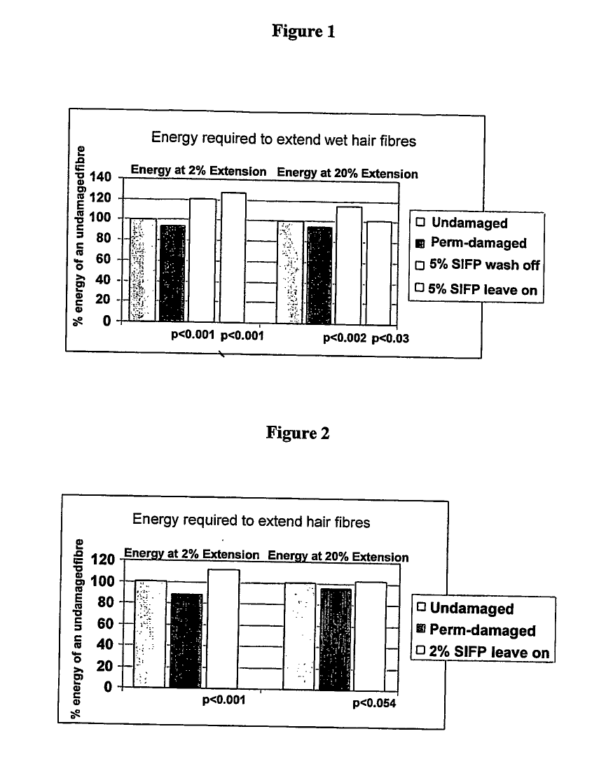

FIG. 1 shows instron test results for permed hair fibres treated with 5% SIFP

FIG. 2 shows instron test results for permed hair fibres treated with 2% SIFP

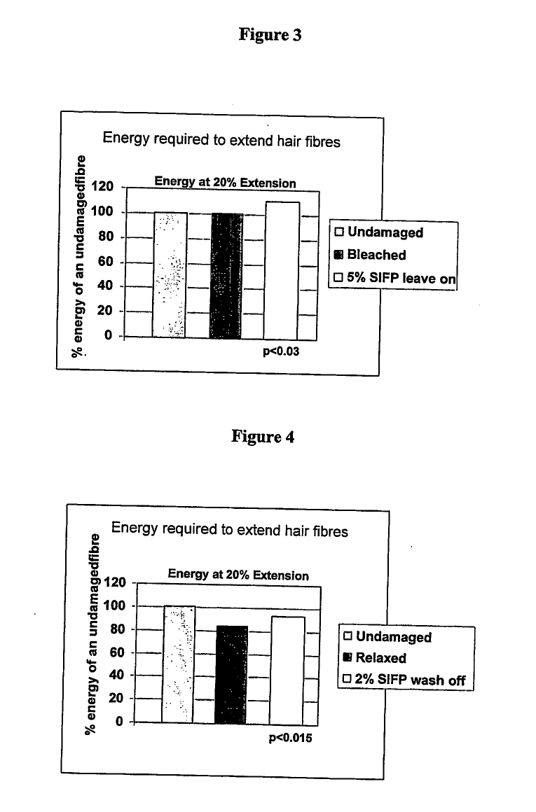

FIG. 3 shows instron test results for bleached hair fibres treated with 5% SIFP

FIG. 4 shows instron results for relaxed hair fibres treated with 2% SIFP

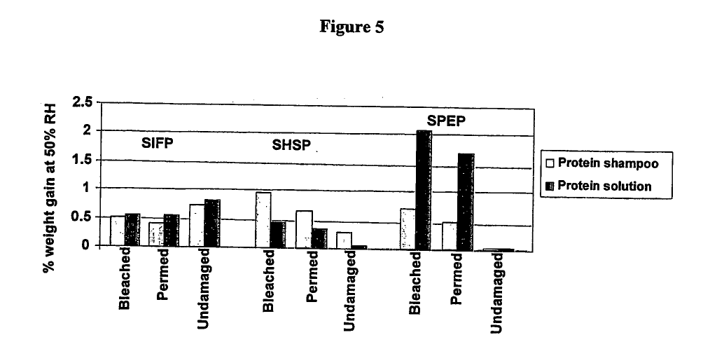

FIG. 5 shows substantivity of SIFP, SHSP and SPEP on undamaged and damaged hair at 50% relative humidity

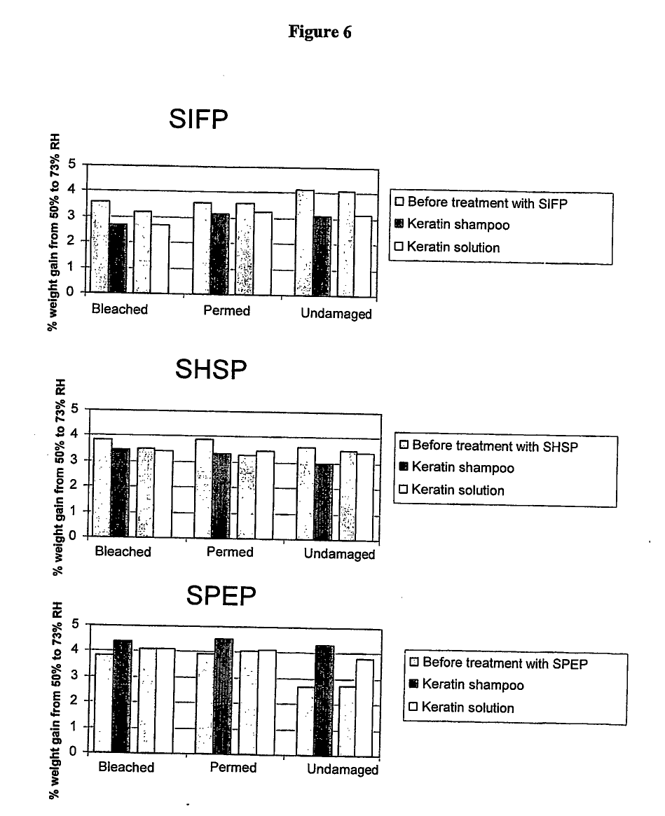

FIG. 6 shows moisturisation with increasing relative humidity of undamaged and damaged hair treated with SIFP, SHSP and SPEP

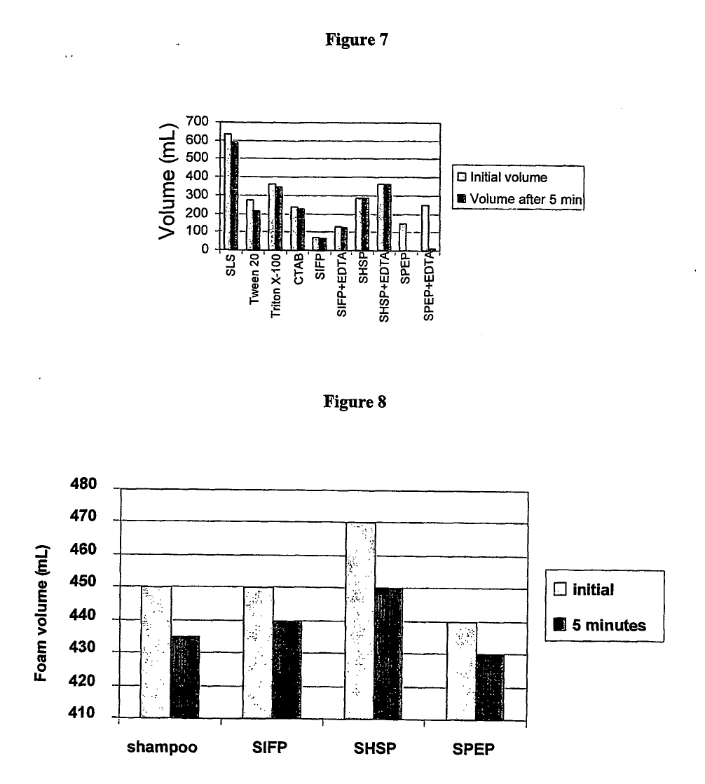

FIG. 7 shows foaming results for common surfactants and SIFP, SHSP and SPEP in the presence and absence of EDTA obtained from the waring blender test

FIG. 8 shows foaming results for shampoo formulations with and without SIFP, SHSP and SPEP

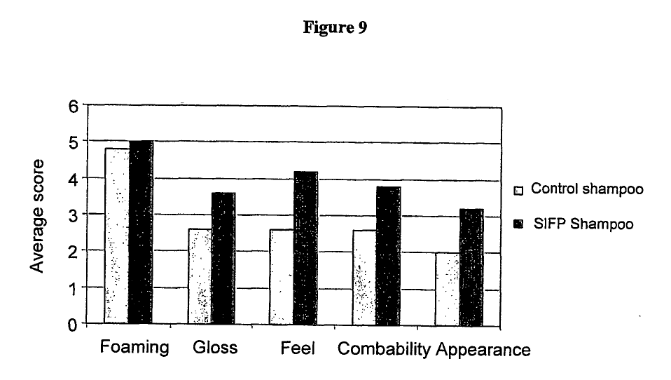

FIG. 9 is a summary of subjective assessment of a shampoo formulation in the presence and absence of SIFP

### DETAILED DESCRIPTION OF THE INVENTION

The hard alpha keratin proteins such as those derived from human hair, wool, animal fibres, horns, hooves or other mammalian sources, can be classified into particular components according to their biochemical properties, specifically their molecular weight and amino acid composition. Table 1 illustrates the amino acid composition determined by conventional analytical methods of typical keratin protein fractions known in the art and also the subject of this invention This involves acid hydrolysis of the analyte which converts all cystine and labile cysteine derivatives to cysteine, typically recorded as half-cysteine.

| | SIFP | SHSP | | | | | |
|---|---|---|---|---|---|---|---|
| | And | And | | | | | |
| | SIFP- | SHSP- | | | | | Whole |
| | pep | pep | SPEP | IFP | HSP | HGTP | wool |
| Cya | 0.4 | 1.7 | 0.7 | 0 | 0 | 0 | 0 |
| Asp | 7.9 | 2.6 | 8 | 9.6 | 2.3 | 3.3 | 5.9 |
| Glu | 15.4 | 8.6 | 15 | 16.9 | 7.9 | 0.6 | 11.1 |
| Ser | 10.9 | 14.3 | 11.4 | 8.1 | 13.2 | 11.8 | 10.8 |
| Gly | 8.1 | 9.1 | 8.4 | 5.2 | 6.2 | 27.6 | 8.6 |
| His | 0.9 | 0.8 | 0.9 | 0.6 | 0.7 | 1.1 | 0.8 |
| Arg | 7.9 | 6.8 | 6.9 | 7.9 | 6.2 | 5.4 | 6.2 |
| Thr | 6.5 | 10.4 | 6.5 | 4.8 | 10.2 | 3.3 | 6.5 |
| Ala | 7.5 | 3.6 | 7.5 | 7.7 | 2.9 | 1.5 | 5.2 |
| Pro | 5.4 | 12.6 | 5.7 | 3.3 | 12.6 | 5.3 | 6.6 |
| Tyr | 1.1 | 1.8 | 1.2 | 2.7 | 2.1 | 15.0 | 3.8 |
| Val | 6.5 | 6.3 | 5.8 | 6.4 | 5.3 | 2.1 | 5.7 |
| Met | 0.2 | 0 | 0.3 | 0.6 | 0 | 0 | 0.5 |
| Lan | 0.2 | 0.2 | 0.3 | 0 | 0 | 0 | 0 |
| Ile | 3.7 | 2.9 | 3.4 | 3.8 | 2.6 | 0.2 | 3 |
| Leu | 8.9 | 3.9 | 8 | 10.2 | 3.4 | 5.5 | 7.2 |
| Phe | 2.5 | 1.5 | 2.1 | 2 | 1.6 | 10.3 | 2.5 |
| Lys | 2.1 | 0.4 | 2.1 | 4.1 | 0.6 | 0.4 | 2.7 |
| Cys | 4.2 | 12.4 | 4.6 | 6 | 22.1 | 6.0 | 13.1 |

Table 1 illustrates an amino acid composition of keratin fractions: S-sulfonated keratin intermediate filament protein (SIFP), peptides derived from S-sulfonated keratin intermediate filament protein (SIFP-pep), S-sulfonated keratin high sulfur protein (SHSP), peptides derived from S-sulfonated keratin high sulfur protein (SHSP-pep), S-sulfonated keratin peptide (SPEP) as used in the invention. Intermediate filament protein (IFP), high sulfur protein (HSP), high glycine-tyrosine protein (HGTP) and whole wool courtesy of Gillespie and Marshall, Variability in the proteins of wool and hair, Proc. Sixth Int. Wool Text. Res. Conf, Pretoria, 2, 67-77, 1980. All residues expressed as mol %. S-sulfocysteine, cystine and cysteine are measured as S-carboxymethyl cysteine following reduction and alkylation, and reported as cys.

Table 2 illustrates the molecular weight determined by conventional analytical methods of typical keratin protein fractions known in the art and also the subject of this invention. Conventional analysis involves cleavage of cystine bonds within the keratin using reduction so that the protein mass is determined in its native, uncrosslinked state, most similar to the unkeratinised state of the protein. Mass is determined using polyacrylamide gel electrophoresis. In the case of the peptide SPEP mass is determined using mass spectrometry. Using these methods the keratin is made soluble without any hydrolysis of peptide bonds and an accurate measure of molecular weight is determined.

TABLE 2
Molecular weight of keratin fractions: S-sulfonated keratin intermediate
filament protein (SIFP), peptides derived from S-sulfonated keratin
intermediate filament protein (SIFP-pep), S-sulfonated keratin high
sulfur protein (SHSP), peptides derived from S-sulfonated keratin
high sulfur protein (SHSP-pep), S-sulfonated keratin peptide (SPEP)
as used in the invention. Intermediate filament protein (IFP), high
sulfur protein (HSP) high glycine-tyrosine protein (HGTP) and whole
wool courtesy of Gillespie and Marshall, Variability in the proteins
of wool and hair, Proc. Sixth Int. Wool Text. Res. Conf., Pretoria,
2, 67-77, 1980.

| Keratin protein fraction | Molecular weight/kD |
|---|---|
| SIFP | 40-60 |
| SHSP | 10-30 |
| SPEP, SIFP-pep, SHSP-pep | <1 |
| IFP | 40-60 |
| HSP | 10-30 |
| HGTP | <10 |

Both amino acid composition and molecular weight varies across keratin types, between species and also within breeds of one species, for example between wools from different breeds of sheep. The figures given in tables 1 and 2 are indicative for the keratin source stated. However, individual types of keratin proteins, or keratin protein fractions, have distinctive characteristics, particularly molecular weight and amino acid content.

The subject of the invention is formulations containing intact S-sulfonated keratin protein fractions. “Intact” refers to proteins that have not been significantly hydrolysed, with hydrolysis being defined as the cleavage of bonds through the addition of water. Gillespie (Biochemistry and physiology of the skin, vol 1, Ed. Goldsmith Oxford University Press, London, 1983, pp 475-510) considers “intact” to refer to proteins in the keratinized polymeric state and further refers to polypeptide subunits which complex to form intact keratins in wool and hair. For the purpose of this invention “intact” refers to the polypeptide subunits described by Gillespie. These are equivalent to the keratin proteins in their native form without the disulfide crosslinks formed through the process of keratinisation.

Keratin protein fractions are distinct groups from within the keratin protein family, such as the intermediate filament proteins, the high sulfur proteins or the high glycine-tyrosine proteins well known in the art. Intermediate filament proteins are described in detail by Orwin et al (Structure and Biochemistry of Mammalian Hard Keratin, Electron Microscopy Reviews, 4, 47, 1991) and also referred to as low sulphur proteins by Gilliespie (Biochemistry and physiology of the skin, vol 1, Ed. Goldsmith Oxford University Press, London, 1983, pp 475-510). Key characteristics of this protein family are molecular weight in the range 40-60 kD and a cysteine content (measured as half cystine) of around 4%. The high sulfur protein family are also well described by Orwin and Gillispie in the same publications. This protein family has a large degree of heterogeity but can be characterised as having a molecular weight in the range 10-30 kD and a cysteine content of greater than 10%. The subset of this family, the ultra high sulfur proteins can have a cysteine content of up to 34%. The high glycine-tryosine protein family are also well described by Orwin and Gillespie in the same publications. This family is also referred to as the high tryrosine proteins and has characteristics of a molecular weight less than 10 kD, a tyrosine content typically greater than 10% and a glycine content typically greater than 20%.

For the purpose of this invention a “keratin protein fraction” is a purified form of keratin that contains predominantly, although not entirely, one distinct protein group as described above. In the context of this invention S-Sulfonated keratins have cysteine/cystine present predominantly in the form S-sulfocysteine, commonly known as the Bunte salt. This highly polar group imparts a degree of solubility to proteins Whilst being stable in solution, the S-sulfo group is a labile cysteine derivative, highly reactive towards thiols, such as cysteine, and other reducing agents Reaction with reducing agents leads to conversion of the S-sulfo cysteine group back to cysteine. S-sulfo cysteine is chemically different to cysteic acid, although both groups contain the SO₃⁻ group. Cysteic acid is produced irreversibly by the oxidation of cysteine or cystine and once formed cannot form disulfide crosslinks back to cysteine. S-sulfocysteine is reactive towards cysteine and readily forms disulfide crosslinks.

One aspect of the invention is personal care formulations containing S-sulfonated keratin intermediate filament protein (SIFP). These proteins are characterised as having a molecular weight in the range 40-60 kD and a cysteine content determined through amino acid analysis of around 4%. This material may be prepared by a variety of methods, including those described in NZ/PCT02/00125. This material has excellent film forming properties, and can be reconstituted in a variety of ways, such as those outlined in NZ/PCT02/00169. The characteristics of the material arise at least in part from the intact nature of the fibrous proteins. Intermediate filament proteins are known to associate on a molecular level, which is fundamental to the reformation of the proteins into materials. The ability of this material to act as a film former is a useful cosmetic property. In addition, the S-sulfo group is of use in personal care formulations as it is highly reactive towards thiols, forming a covalent disulfide bond Thiols are present in the form of cysteine, particularly in hair damaged through reductive processes such as perming. In addition, as a highly polar group, the S-sulfo group is attracted to polar substrates, such as the surface of hair damaged through oxidation processes and bleaching. With this type of hair the SIFP can form salt bridges and hydrogen bonds and consequently impart a durable conditioning effect.

A further aspect of the invention is cosmetic formulations containing S-sulfonated keratin high sulfur protein (SHSP). These proteins are characterised as having a molecular weight in the range 10-30kD and a cysteine content determined through amino acid analysis of greater than 10%. This material may be prepared by a variety of methods, including those described in NZ/PCT02/00125. As an intact globular protein derived from the matrix proteins of the keratin fibre cortex, and also the cuticle cells, this material has the potential to repair damaged hair, in particular where split ends will allow penetration of this intact protein into the fibre. In addition, with a higher proportion of cysteine than commercially available keratin derivatives typically used in personal care formulations, the potential to bind to damaged hair, or to bind to hair when used as part of a permanent waving process, is significant.

One aspect of the invention is keratin peptides derived from keratin protein fractions. These peptides have a cysteine content similar to the fraction from which the peptide is derived (approximately 4% for SIFP-pep and greater than 10% for SHSP-pep). Being of low molecular weight these materials can penetrate the surface of hair and skin and provide cosmetic function within the substrate. This material is differentiated from other hydrolysed keratins by virtue of being derived from a particular keratin protein fraction, as well as the cysteine being present as S-sulfo cysteine. A source of peptides with variable amounts of cysteine is of particular value in the formulation of cosmetics. One aspect of the invention is personal care formulations containing S-sulfonated keratin peptides derived from bulk keratin. These peptides are characterised as having a molecular weight approximately 1 kD or less and a cysteine content determined through amino acid analysis of approximately 4%. This material may be prepared by a variety of methods, including those described in NZ/PCT02/00125. This material is differentiated from other hydrolysed keratins by virtue of the cysteine being present in the form of S-sulfo groups. The low molecular weight of this material allows it to penetrate through the hair cuticle. This feature, combined with the S-sulfo groups present on the peptide and the reactivity of this group creates a useful ingredient for the formulation of cosmetics, in particular hair cosmetics.

Keratins are characterized by having a higher cysteine content than other proteins. In some protein fractions derived from wool cysteine contents as high as 30% have been reported. Cysteine is a known reductant and keratin protein fractions that are the subject of this invention are reductants and antioxidants that can be used as an active component in personal care formulations targeted at anti ageing, or reducing oxidative damage to hair and slin caused by free radicals, pollutants and environmental insults. Measurements of antioxidant properties of keratin protein fractions are detailed in Table 3.

TABLE 3
Antioxidant activity of keratin fractions. Results expressed
as the amount of Trolox equivalent antioxidant capacity per
hundred gram, or milliliters, of sample (μmol TEAC/100 g
or μmol TEAC/100 mL), which represents the amount of Trolox
(vitamin E) that gives the same response as one hundred grams
or mLs, of sample. Triplicate analyses (at different concentrations)
were carried out on each extract. Equivalent activity calculated
on the basis of protein concentration of sample used (SPEP and
SHSP 15% solution, SIFP 5% solution).

| | Antioxidant activity as | | Equivalent activity of | |
|---|---|---|---|---|
| Sample | measured | | 100% protein | |
| SPEP | 281.86 | μmole TEAC/100 mL | 1879 | μmole TEAC/100 mL |
| SIFP | 207.92 | μmole TEAC/100 mL | 4158 | μmole TEAC/100 mL |
| SHSP | 850 | μmole TEAC/100 mL | 5667 | μmole TEAC/100 mL |
| SIFP | 2196 | μmole TEAC/100 g | 2196 | μmole TEAC/100 g |
| powder | | | | |

Personal care formulation includes any substance or preparation intended for placement in contact with any external part of the human body, including the mucous membranes of the oral cavity and the teeth, with a view to:
altering the odours of the body;
changing its appearance;
cleansing it;
maintaining it in good condition; or
perfuming it,

but does not include any product that is required by law to be regulated as a medicine, as a therapeutic substance or device, as a food or as a nutritional or dietary supplement.

It also includes any personal care formulation intended to improve the appearance.

Unless the context clearly requires otherwise, throughout the description and the claims, the words “comprise”, “comprising” and the like, are to be construed in an inclusive sense as opposed to an exclusive or exhaustive sense, that is to say, in the sense of “including, but not limited to”.

The invention will now be described, by way of example only and with reference to the accompanying Examples which are by way of exemplification only.

### EXAMPLES

In each formulation ‘keratin fraction’ is included at an indicative level. Keratin fraction refers to SIFP, SIFP-pep, SHSP, SHSP-pep, HGTP or S-sulfonated keratin peptides, all of which are described above. Unless otherwise stated, it is convenient to provide the keratin fraction in the form of a dilute aqueous solution and include the appropriate amount of this solution in the formulation to achieve the keratin fraction level indicated.

Typical concentrations of aqueous solutions for the keratin fraction types are SIFP 5%, SHSP 15% and S-sulfonated keratin peptides 15%. Therefore, in order to achieve the indicated level of 0.5% keratin fraction for SIFP, 10% of an SIFP solution would to be used in the formulation. Percentages are expressed as w/v.

Sample Formulations

Conditioning Shampoo

| | |
|---|---|
| Sodium lauryl sulphate 28% | 25.0% |
| Sodium laureth-2-sulphate 70% | 4.0 |
| Cocamide DEA 70% | 3.5 |
| Cocamidopropyl betaine (30%) | 3.0 |
| Keratin fraction | 0.5 |
| Sodium chloride | q.s |
| Citric acid | q.s |
| Fragrance | q.s |
| Preservative | q.s |
| Water | q.s to 100 |

Procedure: A. Combine 35.0 g water, sodium laureth sulphate and sodium lauryl sulphate. Heat to 65° C. until dissolved. Add cocamide DEA and allow to cool. B. Mix betaine with water and add to phase A. Add keratin fraction, adjust the pH to 6.5 with citric acid. Add preservative and fragrance as required, adjust to desired thickness with sodium chloride and add remaining water.

Hair Gel

| | |
|---|---|
| Carbomer (Carbopol Ultrez 10) | 0.5% |
| Disodium EDTA | 0.05 |
| Glycerin | 4.0 |
| Triethanolamine (20%) | 3.0 |
| Keratin fraction | 0.45 |
| Preservative | q.s |
| Fragrance | q.s |
| Water | q.s to 100 |

Procedure: A. Heat 60.0 g of water to 70° C. and add to carbopol, EDTA and glycerol. Mix vigorously. Cool. Add triethanolamine to adjust pH to 6.3. Add keratin fraction. Combine preservative and remaining water and add. Mix thoroughly and add fragrance as desired.

Clear Body/Facial Cleanser and Shampoo

| | |
|---|---|
| Ammonium lauryl sulphate 28% | 25.0% |
| Disodium laureth sulfosuccinate | 20.0 |
| Cocamidopropyl betaine | 8.0 |
| Keratin fraction | 0.5 |
| Sodium chloride | qs |
| Fragrance (parfum) | qs |
| Preservative | qs |
| Water (aqua) | qs to 100 |

Hair Conditioner

| | |
|---|---|
| Cetrimonium chloride | 5.0% |
| Stearyl alcohol | 4.5 |
| Keratin fraction | 0.25 |
| Fragrance | qs |
| Preservative | qs |
| Water | qs to 100 |

Hair Mousse

| | |
|---|---|
| Keratin fraction | 0.25% |
| Hydrogenated tallow trimonium chloride | 0.20 |
| Nonoxynol-10 | 0.35 |
| Alcohol | 10.0 |
| Butane-48 | 10.0 |
| Water | qs to 100 |

Setting Lotion

| | |
|---|---|
| Carbomer (Carbopol Ultrez 10) | 2.0% |
| Mineral oil (light) | 0.20 |
| Keratin fraction | 0.25 |
| Alcohol | 37.5 |
| Fragrance | qs |
| Water | qs to 100 |

Hairspray

| | |
|---|---|
| VA/Crotonates/Vinyl Neodeconoate Copolymer | 1.60% |
| (Resyn 28-2930) | |
| Aminomethyl propanol | 0.15 |
| PEG-75 lanolin | 0.20 |
| Keratin fraction | 0.25 |
| Alcohol | 65.05 |
| Butane 30 | 28.0 |

Pre-Perming Solution

| | |
|---|---|
| TEA lauryl sulphate | 30.0% |
| Cocamidopropyl dimethylamine oxide | 10.0 |
| Cocamide DEA | 7.5 |
| Cocamidopropyl betaine | 20.0 |
| Cocamide MEA | 3.0 |
| Keratin fraction | 0.5 |
| Fragrance | qs |
| Preservative | qs |
| Water | qs |

Post-Perming Solution

| | |
|---|---|
| Keratin fraction | 0.5% |
| Cocamidopropyl dimethylamine oxide | 10.0 |
| PPG-5-ceteth-10-phosphate | 0.5 |
| Glycerin | 3.0 |
| Hydroxypropyl methylcellulose | 1.5 |
| Fragrance | qs |
| Preservative | qs |
| Water | qs to 100 |

Moisturising Cream

| | |
|---|---|
| Cetearyl alcohol and ceteareth-20 | 5.0% |
| Cetearyl Alcohol | 2.0 |
| Mineral oil (light) | 5.0 |
| Keratin fraction | 0.5 |
| Preservative | 0.3 |
| Fragrance | q.s |
| Water | q.s to 100 |

Hand and Body Lotion

| | |
|---|---|
| Polyglyceryl-3 methylglucose distearate | 4.0% |
| Stearyl/behenyl beeswaxate | 3.0 |
| Octyldodecanol | 4.0 |
| Avocado oil | 6.0 |
| Mineral oil | 3.0 |
| Jojoba oil | 2.0 |
| Keratin fraction | 0.5 |
| Ceramide III | 0.2 |
| Propylene glycol | 3.0 |
| Preservative | q.s. |
| Fragrance (Parfum) | q.s |
| Water (aqua) | q.s. to 100 |

Anti-Wrinkle Treatment Cream

| | |
|---|---|
| Sodium behenoyl lactylate | 2.0% |
| Cetearyl alcohol | 3.0 |
| Glyceryl stearate | 2.6 |
| Isopropyl palmitate | 6.0 |
| Sunflower seed oil | 6.0 |
| Keratin fraction | 0.5 |
| Glycerine | 3.0 |
| Magnesium ascorbyl phosphate (and) lecithin | 6.0 |
| (Rovisome-C, R.I.T.A) | |
| Preservative | q.s. |
| Water | q.s. to 100 |

Facial Moisture Cream

| | |
|---|---|
| Myristyl lactate | 3.0% |
| Laneth-25 (and) ceteth-25 (and) oleth-25 (and) | 1.0 |
| Steareth-25 (Solulan 25, Amerchol) | |
| Mineral oil (70 visc.) | 16.5 |
| Petrolatum | 3.0 |
| Tocotrienol | 1.0 |
| Carbomer 934 | 0.75 |
| Keratin fraction | 0.5 |
| Triethanolamine (10% aq.) | 7.5 |
| Preservative | q.s |
| Fragrance | q.s. |
| Water | q.s. to 100 |

Moisturising Body Lotion

| | |
|---|---|
| Methyl glucose dioleate | 2.0% |
| Methyl glucose sesquistearate | 1.5 |
| Methyl gluceth-20 distearate | 1.5 |
| Cetearyl alcohol (and) ceteareth-20 | 1.5 |
| Isopropyl palmitate | 3.0 |
| Ceramide 3, hexyldecanol | 2.0 |
| Methyl gluceth-10 | 3.0 |
| Keratin fraction | 0.5 |
| Carbomer 1342 | 0.2 |
| Triethanolamine | 0.2 |
| Fragrance | q.s. |
| Preservative | q.s. |
| Water | q.s to 100 |

Cationic Emollient Lotion

| | |
|---|---|
| Isostearamidopropyl laurylacetodimonium chloride | 5.0% |
| Lactamide MEA | 3.0 |
| Isostearyl neopentanoate | 15.0 |
| Myristyl myristate | 1.0 |
| Cetyl alcohol | 4.0 |
| Glyceryl isostearate | 3.5 |
| Keratin fraction | 0.5 |
| Preservative | q.s. |
| Water | q.s. to 100 |

Men's Facial Conditioner

| | |
|---|---|
| Carbomer (Ultrez 10 Carbopol) | 0.4% |
| Propylene glycol | 1.0 |
| PPG-5-buteth | 0.5 |
| Beta glucan | 2.0 |
| PEG-60 hydrogenated castor oil | 0.5 |
| Triethanolamine (99%) | 0.4 |
| Keratin fraction | 0.5 |
| SD-39 C alcohol (Quantum) | 5.0 |
| Fragrance | q.s. |
| Preservative | q.s. |
| Water | q.s. to 100 |

Moisturising After Shave Treatment

| | |
|---|---|
| Ceteareth-12 (and) ceteareth-20 (and) cetearyl | 6.0% |
| alcohol (and) cetyl palmitate (and) glyceryl | |
| stearate (Emulgade SE, Henkel) | |
| Cetearyl alcohol | 1.0 |
| Dicaprylyl ether | 8.0 |
| Octyldodecanol | 4.0 |
| Glycerin | 3.0 |
| Carbomer (Ultrez 10 Carbopol) | 0.3 |
| Keratin fraction | 0.5 |
| Bisabolol | 0.2 |
| Ethyl alcohol | 3.0 |
| Water (and) sodium hyaluronate, (and) wheat | 4.0 |
| (triticum vulgare) germ extract (and) saccharomyces | |
| (and) cerevisiae extract (Eashave, Pentapharm) | |
| Triethanolamine | q.s. |
| Fragrance | q.s. |
| Preservative | q.s. |
| Water | q.s. to 100 |

Antioxidant Cream

| | |
|---|---|
| Glycerin (99.7%) | 3.0% |
| Xanthan gum | 0.15 |
| Disodium EDTA | 0.05 |
| Hydrogenated polyisobutene | 1.0 |
| Isopropyl palmitate | 5.0 |
| Petrolatum | 0.75 |
| Dimethicone | 0.75 |
| Cyclopentasiloxane | 3.0 |
| Steareth-2 | 1.0 |
| PEG-100 stearate | 1.9 |
| Cetyl alcohol | 2.0 |
| Ethylhexyl palmitate | 3.0 |
| Polyacrylamide (and) C13-14 isoparaffin (and) | 2.0 |
| laureth-7 (sepigel 305, Seppic) | |
| Keratin fraction | 0.5 |
| Glycerin (and) water (and) vitis vinitera (grape) | 0.5 |
| seed extract (Collaborative) | |
| Fragrance | q.s. |
| Preservative | q.s. |
| Water | q.s. to 100 |

Liquid Detergent

| | |
|---|---|
| Sodium laureth sulphate | 50.0% |
| Cocamide DEA | 3.0 |
| Keratin fraction | 0.25 |
| Sodium chloride | qs |
| Preservative | qs |
| Citric acid | qs |
| Water | qs to 100 |

Shower Gel

| | |
|---|---|
| Sodium laureth sulphate | 35.0% |
| Sodium lauroyl sarcosinate | 5.0 |
| Cocoamidopropyl betaine | 10.0 |
| Cocoamidopropyl hydroxyl sultaine | 5.0 |
| Glycerine | 2.0 |
| Keratin fraction | 0.15 |
| Tetrasodium EDTA | 0.25 |
| Citric acid | qs |
| Fragrance | qs |
| Preservative | qs |
| Water | qs to 100 |

Foaming Bath Gel

| | |
|---|---|
| TEA lauryl sulphate | 40.0% |
| Lauroyl diethanolamide | 10.0 |
| Linoleic diethanolamide | 7.0 |
| PEG-75 lanolin oil | 5.0 |
| Keratin fraction | 0.25 |
| Tetrasodium EDTA | 0.5 |
| Fragrance | qs |
| Preservative | qs |
| Dyes | qs |
| Water | qs to 100 |

Nail Polish

For this example it is convenient to provide the keratin fraction as a dry powder, in the form of the S-sulfonic acid.

First Coat

| | |
|---|---|
| Keratin fraction (SIFP) | 10.0% |
| Sodium hydroxide (4%) | 10.0 |
| Keratin fraction (SHSP or SPEP) | qs |
| Sodium lauryl sulphate | qs |
| Dye or Pigment | qs |
| Water | qs to 100 |

Nail Glosser

| | |
|---|---|
| Keratin fraction (SIFP) | 10.0% |
| Keratin fraction (SHSP or sulfonated keratin peptide) | qs |
| Sodium hydroxide (4%) | 10.0 |
| Sodium lauryl sulphate | qs |
| Water | qs to 100 |

Hardener

| | |
|---|---|
| Citric acid | 21.0% |
| Water | 79.0 |

Mascara

| | |
|---|---|
| PEG-8 | 3.0% |
| Xanthan gum | 0.50 |
| Tetrahydroxypropyl ethylenediamine | 1.3 |
| Carnauba wax | 8.0 |
| Beeswax | 4.0 |
| Isoeicosane | 4.0 |
| Polyisobutene | 4.0 |
| Stearic acid | 5.0 |
| Glyceryl stearate | 1.0 |
| Keratin fraction | 0.25 |
| Pigments | 10.0 |
| Polyurethane-1 | 8.0 |
| VP/VA Copolymer | 2.0 |
| Preservative | qs |
| Fragrance | qs |
| Water | qs to 100 |

Liquid Foundation

| | |
|---|---|
| Polysorbate 80 | 0.1% |
| Potassium hydroxide | 0.98 |
| Keratin fraction | 0.25 |
| Titanium dioxide/talc, 80% | 0.1 |
| Talc | 3.76 |
| Yellow iron oxide/talc, 80% | 0.8 |
| Red iron oxide/talc, 80% | 0.38 |
| Black iron oxide/talc, 80% | 0.06 |
| Propylene glycol | 6.0 |
| Magnesium aluminum silicate | 1.0 |
| Cellulose gum | 0.12 |
| di-PPG-3 myristyl ether adipate | 12.0 |
| Cetearyl alcohol (and) ceteth-20 phosphate (and) | 3.0 |
| dicetyl phosphate (Crodafos CS 20 Acid) | |
| Steareth-10 | 2.0 |
| Cetyl alcohol | 0.62 |
| Steareth-2 | 0.5 |
| Preservative | qs |
| Water | qs to 100 |

Shaving Cream

| | |
|---|---|
| Sodium cocosulfate | 5.0% |
| Keratin fraction | 0.25 |
| Glycerin | 7.0 |
| Disodium lauryl sulfosuccinate | 50.0 |
| Disodium EDTA | qs |
| Sodium chloride | qs |
| Citric acid | qs |
| Fragrance | qs |
| Preservative | qs |
| Water | qs to 100 |

Lipstick

| | |
|---|---|
| Octyldodecanol | 22.0% |
| Oleyl alcohol | 8.0 |
| Keratin fraction | 0.16 |
| C30-45 alkyl methicone | 20.0 |
| Lanolin oil | 14.0 |
| Petrolatum | 5.0 |
| Bentone 36 (Rheox) | 0.6 |
| Tenox 20 (Eastman) | 0.1 |
| Pigment/castor oil | 10.0 |
| Preservative | qs |
| Cyclomethicone | qs to 100 |

Sulfite Hair Straightener

| | |
|---|---|
| Carbomer (Carbopol 940) | 1.5% |
| Ammonium bisulphate | 9.0 |
| Diethylene urea | 10.0 |
| Cetearth 20 | 2.0 |
| Keratin fraction | 0.5 |
| Fragrance | qs |
| Ammonium hydroxide 28% | qs to pH 7.2 |
| Water | qs to 100 |

Post Straightening Neutralising Solution

| | |
|---|---|
| Sodium bicarbonate | 2.35% |
| Sodium carbonate | 2.94 |
| EDTA | 0.15 |
| Cetearth 20 | 0.2 |
| Keratin fraction | 0.5 |
| Fragrance | qs |
| Water | qs to 100 |

Pre-Relaxer Conditioner

| | |
|---|---|
| Cationic polyamine | 2.0% |
| Imidazolidinyl urea | 0.25 |
| Keratin fraction | 0.5 |
| Fragrance | qs |
| Preservative | qs |
| Water | qs to 100 |

Alkali Metal Hydroxide Straightener (Lye)

| | |
|---|---|
| Bentonite | 1.0% |
| Sodium Lauryl Sulphate | 1.5 |
| PEG-75 lanolin | 1.5 |
| Petrolatum | 12.0 |
| Cetearyl alcohol | 12.0 |
| Sodium hydroxide | 3.1 |
| Keratin fraction | 0.5 |
| Fragrance | qs |
| Water | qs to 100 |

Post Relaxing Shampoo

| | |
|---|---|
| Sodium lauryl sulphate | 10.0% |
| Cocamide DEA | 3.0 |
| EDTA | 0.2 |
| Keratin fraction | 0.5 |
| Citric acid | qs to pH 5.0 |
| Fragrance | qs |
| Preservative | qs |
| Water | qs to 100 |

Hair Tonic/Cuticle Cover

| | |
|---|---|
| Glycerine | 5.5% |
| EDTA | 0.07 |
| Carbomer (Carbopol Ultrez 10) | 0.33 |
| Triethanolamine (20%) | 1.0 |
| Keratin fraction | 0.5 |
| Ethanol | 10.0 |
| Preservative | qs |
| Water | qs to 100 |

Leave In Hair Conditioner

| | |
|---|---|
| Cetyl alcohol | 5.0% |
| Glyceryl stearate | 3.0 |
| Petrolatum | 0.7 |
| Isopropyl myristate | 1.5 |
| Polysorbate 60 | 1.0 |
| Dimethiconol & cyclomethicone | 4.0 |
| Glycerine | 7.0 |
| EDTA | 0.1 |
| D-panthenol | 0.2 |
| Keratin fraction | 0.5 |
| Cyclomethicone | 4.0 |
| Fragrance | qs |
| Preservative | qs |
| Water | qs to 100 |

Post Hair-Dyeing Conditoner

| | |
|---|---|
| Quaternium-40 | 2.0% |
| Keratin fraction | 0.5 |
| Amphoteric-2 | 4.0 |
| Hydroxyethyl cellulose | 2.0 |
| Phosphoric acid | qs to pH 4.5 |
| Fragrance | qs |
| Water | qs to 100 |

Temporary Hair Colouring Styling Gel

| | |
|---|---|
| Dimethicone copolyol | 1.5% |
| PPG-10 methyl glucose ether | 1.0 |
| Polyvinylpyrrolidone | 2.5 |
| Triisopropanolamine | 1.1 |
| Carbomer (Carbopol 940) | 0.6 |
| Laureth-23 | 1.0 |
| Phenoxyethanol | 0.2 |
| Keratin fraction | 0.5 |
| EDTA | 0.01 |
| D&C orange 4 | 0.12 |
| Ext D&C Violet 2 | 0.02 |
| FD&C yellow 6 | 0.02 |
| Ethanol | 5.0 |
| Fragrance | qs |
| Water | qs to 100 |

Formulations containing keratin fractions may improve the cosmetic properties of hair. This is illustrated by the following examples.

### Example 1

### Strengthening

Instron Method

Hair fibres placed in water prior to measurement with Instron tensile tester. Load cell 10N, Load range 10%, speed 30 mm/min, gauge length 15 mm.

Energy required to extend individual hair fibres by 2% and 20% was recorded for 50 fibres and averaged.

Materials

Perming solution 8% thioglycollic acid, pH adjusted to 8 with ammonia solution.

Perming Neutraliser 2.5% hydrogen peroxide

Bleaching Solution 9% hydrogen peroxide, 1% ammonium persulfate, pH 8.3

Hair straightening (relaxing) solution 2.5% sodium hydroxide

Relaxer Neutraliser 9.5% citric acid

Perming Protocol

1. Hair fibres (˜4 cm in length) from the same source (Caucasian) were immersed in perming solution for 3 hours.

2. Placed in the neutralising solution for 30 min and air dried.

3. Placed in a solution containing the appropriate amount of keratin fraction for 30 min.

4. Treated fibres were rinsed, dried and equilibrated at 50% relative humidity, 23° C. overnight in the case of the “wash off” procedure. The rinsing step was omitted in the case of the “leave on” procedure.

5. Energy required to extend measured on Instron apparatus.

Bleaching Protocol

1. Hair fibres (˜4 cm in length) from the same source (Caucasian) were immersed in bleaching solution for 3 hours.

2. Placed in a solution containing the appropriate amount of keratin fraction for 30 min.

3. Rinsed, dried and equilibrated at 50% relative humidity, 23° C. overnight.

4. Energy required to extend measured on Instron apparatus.

Relaxing Protocol

1. Hair fibres (˜4 cm in length) from the same source (Caucasian) were immersed in relaxing solution for 30 min.

2. Placed in the neutralising solution for 5 min, rinsed in RO water and air dried.

3. Placed in a solution containing the appropriate amount of keratin fraction for 30 min.

4. Rinsed, dried and equilibrated at 50% relative humidity, 23° C. overnight.

5. Energy required to extend measured on Instron apparatus.

### Test Example 1

Perming protocol used with keratin fraction of 5% SIFP (supplied as a 5% aqueous solution) i.e. 0.25% active. Instron tensile tester method as described previously. Results are shown in Table 4 and FIG. 1.

TABLE 4
Instron test results for permed and undamaged hair
fibres treated with 5% SIFP. Results expressed as average
energy (millijoules) required to extend hair fibres
by 2 and 20% of the gauge length (15 mm).

| | Average | | Average | |
|---|---|---|---|---|
| | Energy at 2% | Students t test | Energy at 20% | |
| Description | (mJ) | (p) | (mJ) | p |
| Undamaged | 0.0406 | | 3.718 | |
| Permed | 0.0382 | | 3.543 | |
| Wash | 0.0491 | <0.001 | 4.030 | <0.02 |
| Leave on | 0.0515 | <0.001 | 3.871 | <0.03 |

This study indicates that hair fibres which have been weakened by a perming process regain strength following treatment with a solution containing a keratin fraction in both wash off and leave on protocols. The increase in energy needed to extend the permed/keratin treated fibres relative to the permed fibres was measured statistically using the student's t test and found to be significant in all cases.

### Test Example 2

Perming protocol used with keratin fraction of 2% SIFP (supplied as a 5% aqueous solution) i.e. 0.1% active. Instron tensile tester method as described previously. Results are shown in Table 5 and FIG. 2.

TABLE 5
Instron test results for permed and undamaged hair
fibres treated with 2% SIFP. Results expressed as average
energy (millijoules) required to extend hair fibres
by 2 and 20% of the gauge length (15 mm).

| | Average | | Average | |
|---|---|---|---|---|
| Description | Energy at 2% | p | Energy at 20% | p |
| Undamaged | 0.0316 | | 3.252 | |
| Permed | 0.0278 | | 3.100 | |
| Leave on | 0.0357 | <0.001 | 3.325 | <0.054 |

This study shows that permed hair fibres are strengthened after treatment with a 0.1% active solution of keratin fraction when it is used as part of a leave on protocol. The difference was analysed statistically using the Student's t test and found to be statistically significant (p<0.001 at 2% extension and p<0.054 at 20% extension).

### Test Example 3

Bleaching protocol used with keratin fraction of 5% SIFP (supplied as a 5% aqueous solution) i.e. 0.25% active. Instron tensile tester method as described previously. Results are shown in Table 6 and FIG. 3.

TABLE 6
Instron test results for bleached and undamaged hair fibres treated
with 5% SIFP. Results expressed as average energy (millijoules) required
to extend hair fibres by 20% of the gauge length (15 mm).

| | Average | |
|---|---|---|
| Description | Energy at 20% | p |
| Undamaged | 3.610 | |
| Bleached | 3.610 | |
| Leave on | 4.004 | <0.03 |

This study indicates that hair fibres which have been subjected to bleaching have increased strength following treatment with a solution containing 0.25% active keratin protein fraction as part of a leave on protocol. The difference was analysed statistically using the Student's t test and found to be statistically significant (p<0.03).

### Test Example 4

Relaxing protocol used with keratin fraction of 2% SIFP (supplied as a 5% aqueous solution) i.e. 0.1% active. Instron tensile tester method as described previously. Results are shown in Table 7 and FIG. 4.

TABLE 7
Instron test results for relaxed and undamaged hair fibres treated
with 2% SIFP. Results expressed as average energy (millijoules) required
to extend hair fibres by 20% of the gauge length (15 mm).

| | Average | |
|---|---|---|
| Description | Energy at 20% | P |
| Undamaged | 3.610 | |
| Relaxed | 2.997 | |
| Wash off | 3.378 | <0.015 |

This study indicates that hair fibres which have been subjected to a hair straighteneing procedure have increased strength following treatment with a solution containing 0.1% active keratin protein fraction as part of a wash off protocol. The difference was analysed statistically using the Student's t test and found to be statistically significant p<0.015).

Test examples 1-4 demonstrate the keratin protein fractions impart a strengthening effect (as measured by an increase in the energy required to extend individual hair fibres) on hair which has been subjected to perming, bleaching and straightening which are routinely used cosmetic treatments.

### Example 2

### Substantivity

Keratin Shampoo Formulation

| | % by weight |
|---|---|
| Ammonium lauryl sulphate (28%) | 25.0 |
| Disodium laureth sulfosuccinate | 20.0 |
| Cocamidopropyl betaine | 8.0 |
| Preservative | 0.3 |
| Keratin fraction | 0.5 |
| Sodium chloride (20%) | q.s |
| Water | q.s to 100 |

Experimental Procedure

Hair swatches 2-3 g were used. Experiments were performed in duplicate. Swatches were shampooed prior to use to remove residual conditioning agents. Swatches were either left undamaged, or were subjected to multiple perming procedures or bleaching procedures.

Swatches were equilibrated at 50% RH and weighed accurately. Keratin fractions were applied to the swatches either from an aqueous solution or as part of a shampoo formulation at a level of 3.0 ml per swatch.

The treatment solution was spread onto the swatch with fingertips, allowed to absorb for 1 min and rinsed under a stream of RO water.

The swatch was air-dried and equilibrated at 50% RH for 24 hr prior to weighing.

Results are summarized in Table 8 and FIG. 5.

TABLE 8
Percentage weight gain at 50% relative humidity for damaged
and undamaged hair with and without treatment with a solution
or shampoo formulation containing SIFP, SHSP and SPEP.

| | Average weight gain (%) at 50% Relative Humidity | | |
|---|---|---|---|
| Keratin fraction | Description | Shampoo | Solution |
| SIFP | Bleached | 0.51 | 0.56 |
| | Permed | 0.41 | 0.55 |
| | Undamaged | 0.74 | 0.82 |
| SHSP | Bleached | 0.96 | 0.46 |
| | Permed | 0.66 | 0.35 |
| | Undamaged | 0.28 | 0.06 |
| SPEP | Bleached | 0.72 | 2.10 |
| | Permed | 0.50 | 1.70 |
| | Undamaged | 0.0 | 0.0 |

This study indicates that the SIFP keratin fraction is substantive to undamaged, permed and bleached hair from both an aqueous solution and shampoo formulation. The SHSP keratin fraction is also substantive from an aqueous solution and shampoo formulation and seems to adsorb to a greater extent to bleached and permed hair and when applied as a solution rather than a shampoo. The keratin fraction which has molecular weight less than 1 kD, SPEP, is substantive to bleached and permed hair from an aqueous solution and shampoo however it was not associated with a weight increase on undamaged hair. A much greater weight increase was observed from an aqueous solution indicating that the surfactants present in the shampoo may be removing the keratin fraction.

These results indicate that the different keratin fractions have different surface activity on the hair fibre. The larger fractions have a greater ability to form adsorbing layers and convey a conditioning and smoothing (gloss) effect compared with the low molecular weight SPEP.

### Example 3

### Moisturisation

Experimental Procedure

Hair swatches 2-3 g were used. Each treatment within the experiment was performed in duplicate.

Swatches were shampooed with a high surfactant (non-conditioning) shampoo prior to use to remove residual conditioning agents.

Swatches were either left undamaged, or were subjected to multiple perming or bleaching procedures.

Swatches were equilibrated at 50% RH for 24 hrs and weighed accurately.

Swatches were equilibrated at 73% RH for 24 hrs and weighed accurately.

The difference in weight as a result of increased humidity (in the absence of protein treatment) was calculated.

Swatches were treated (in duplicate) with either an aqueous solution containing a keratin fraction or a shampoo containing a keratin fraction (as described earlier).

Swatches were equilibrated for 24 hrs and weighed at 50% RH.

Swatches were equilibrated for 24 hr and weighed at 73% RH.

The difference in weight as a result of increased humidity following treatment with a keratin solution or shampoo was calculated.

Results are summarized in Table 9 and FIG. 6.

TABLE 9
Percentage weight increase with increasing relative humidity
for damaged and undamaged hair fibres treated with an aqueous
solution or a shampoo containing SIFP, SHSP or SPEP.

| | % weight increase due to moisture uptake | | | | |
|---|---|---|---|---|---|
| | on going from 50 to 73% RH | | | | |
| | | Pre- | | Pre- | |
| Keratin | | Protein | Protein | Protein | Protein |
| Fraction | Description | shampoo | shampoo | solution | solution |
| SIFP | Bleached | 3.6 | 2.7 | 3.2 | 2.7 |
| | Permed | 3.6 | 3.15 | 3.6 | 3.25 |
| | Undamaged | 4.15 | 3.1 | 4.1 | 3.15 |
| SHSP | Bleached | 3.85 | 3.45 | 3.5 | 3.4 |
| | Permed | 3.9 | 3.35 | 3.3 | 3.45 |
| | Undamaged | 3.65 | 3.0 | 3.5 | 3.4 |
| SPEP | Bleached | 3.85 | 4.4 | 4.1 | 4.1 |
| | Permed | 3.95 | 4.55 | 4.05 | 4.1 |
| | Undamaged | 2.7 | 4.3 | 2.75 | 3.8 |

This study indicates moisturisation could be increased or decreased depending on the keratin fraction applied. The SIFP keratin fraction decreased moisture uptake of permed, bleached and undamaged hair at high humidity when applied as an aqueous solution or in a shampoo.

The SHSP fraction had less of an effect on moisture uptake at high humidity and there was some indication that moisturisation decreased when applied from a shampoo in preference to an aqueous solution.

SPEP increased moisture uptake particularly when applied from a shampoo.

### Example 4

### Foaming of Formulations

Experimental Procedure

Waring Blender Test Method:
1. Prepare 100 mL of a 5% solution of material to be tested.
2. Pour into blender.
3. Blend for 1 minute on high.
4. Pour all the liquid into a 500 mL measuring cylinder.
5. Record the amount of foam (−100 mL) immediately and record.
6. Record the amount of foam in mLs after 5 minutes: (this will give “low foam” measurement.)

### Test Example 7

Comparison of foaming of keratin fraction with common surfactants and effect of adding 0.5% metal ion sequesterant ethylenediammine tetraacetic acid (EDTA).

Waring blender test applied.

Results are summarized in Table 10 and FIG. 7.

TABLE 10
Foam quantity and stability in a waring blender
test. Results are expressed as foam volume immediately
following blending and after 5 minutes.

| Description | Initial volume(ml) | Volume after 5 min |
|---|---|---|
| Sodium lauryl sulphate | 635 | 595 |
| (SLS) | | |
| Tween 20 | 275 | 215 |
| Triton X-100 | 365 | 345 |
| CTAB | 240 | 230 |
| SIFP | 70 | 65 |
| SIFP + EDTA | 130 | 125 |
| SHSP | 285 | 285 |
| SHSP + EDTA | 365 | 365 |
| SPEP | 150 | 0 |
| SPEP + EDTA | 250 | 10 |

This study indicates that the SIFP keratin fraction shows mild foaming and forms stable foams. The SHSP fraction displayed intermediate foaming ability and formed very stable foams. SPEP formed unstable foams. The addition of the ion sequestering agent EDTA increased the foaming capacity of all fractions.

### Test Example 8

Foaming properties of keratin fraction mixtures.

Keratin fractions were combined and the waring blender test used to assess foaming.

Results are summarised in Table 11.

TABLE 11
Foam quantity and stability of keratin fraction mixtures
in a waring blender test. Results are expressed as foam volume
immediately following blending and after 5 minutes.

| Description | Initial volume (mL) | Volume after 5 min (ml) |
|---|---|---|
| 4% SIFP, 1% SHSP | 220 | 210 |
| 2.5% SIFP, 2.5% SHSP | 175 | 165 |
| 1% SIFP, 4% SHSP | 120 | 110 |

This study indicates that addition of the SHSP keratin fraction to the less foaming SIFP fraction increases the foam capacity.

### Test Example 9

Foaming of shampoo formulations containing keratin fractions.

Shampoo formulation described earlier, containing 0.5% active keratin fraction.

Waring blender test results summarized in Table 12 and FIG. 8.

TABLE 12
Foam quantity and stability of shampoo with and without SIFP, SHSP
and SPEP in a waring blender test. Results are expressed as foam
volume immediately following blending and after 5 minutes.

| Description | Initial volume (ml) | Volume after 5 min (ml) |
|---|---|---|
| Shampoo only | 450 | 435 |
| SIFP shampoo | 450 | 440 |
| SHSP shampoo | 470 | 450 |
| SPEP shampoo | 440 | 430 |

It is known that proteins often have an adverse effect of foaming in formulations. This study indicates that addition of the SIFP keratin fraction to a shampoo formulation does not have a deleterious effect on foaming, moreover there is some evidence that foam stability in increased. Furthermore addition of the SHSP fraction to a shampoo formulation increases the foaming capacity and results in a greater foam after 5 minutes compared to that in the absence of the keratin. The SPEP keratin fraction does suppress foam formation.

### Example 5

### Subjective Assessment of Keratin Fractions in Shampoo Formulation

Method

Human volunteers were given two unlabelled shampoo formulations (described earlier), one of which contained 0.5% active of the SIFP keratin fraction.

Volunteers were asked to wash their hair with one sample as many times as usual over the period of one week and then repeat with the other sample.

Volunteers were then given a questionnaire to fill out ranking each sample in terms of foaming ability, gloss impartment, hair feel, combablility, and appearance.

The lower number was associated with an undesirable effect eg in the case of combability 1=extremely difficult to comb and 6=excellent combability.

### Test Example 10

Questionnaires were collected and the scores recorded and averaged.

Results are summarized in Table 13 and FIG. 9.

TABLE 13
Subjective assessment of a shampoo formulation
with and without SIFP. Results are an average
of scores recorded by human volunteers.

| | Shampoo only (average | Shampoo + SIFP keratin |
|---|---|---|
| Attribute | score) | fraction (average score) |
| Foaming | 4.8 | 5.0 |
| Gloss | 2.6 | 3.6 |
| Feel | 2.6 | 4.2 |
| Combability | 2.6 | 3.8 |
| Appearance | 2.0 | 3.2 |

This study indicates that volunteers did not observe a major change in foaming of the shampoo formulation as a result of addition of the keratin fraction. Moreover the presence of the keratin fraction was observed to impart superior gloss, feel, combability and improved appearance to the formulation indicating that it was acting as a conditioning agent.

Whilst the invention has been described with reference to the above Examples, it will be appreciated that numerous improvements and modifications may be made without departing from the scope of the invention as set out in this specification.

### INDUSTRIAL APPLICABILITY

The compositions described in the application will be useful in a wide range of personal care products such as shampoos, gels, conditioners, creams and detergents and including cosmetics such as moisturizers, lotions, creams and gels.

1. A personal care formulation containing an s-sulfonated keratin protein fraction.

2. A personal care formulation as claimed in claim 1 wherein the keratin protein fraction is intact.

3. A personal care formulation as claimed in claim 1 wherein the keratin protein fraction is hydrolysed.

4. A personal care formulation as claimed in claim 1, wherein the keratin protein fraction is from the intermediate filament protein family.

5. A personal care formulation as claimed in claim 1 wherein the keratin protein fraction is from the high sulfur protein family.

6. A personal care formulation according to claim 4 wherein the cysteine content of the keratin protein is around 4%.

7. A personal care formulation according to claim 5 wherein the cysteine content of the keratin protein is greater than 10%.

8. A personal care formulation as claimed in claim 1, wherein the keratin protein fraction is from the high glycine-tyrosine protein family.

9. A personal care formulation containing from about 0.001% to 50% of an s-sulfonated keratin protein fraction.

10. A personal care formulation according to claim 9 containing from 0.001% to 10% of an s-sulfonated keratin protein fraction.

11. A personal care formulation according to claim 10 containing from 0.001% to 1% of an s-sulfonated keratin protein fraction.

12. A personal care formulation according to claim 9 wherein the keratin protein fraction is intact.

13. A personal care formulation according to claim 9 wherein the keratin protein fraction is hydrolysed.

14. A personal care formulation according to claim 12 wherein the keratin protein fraction is from the intermediate filament protein family.

15. A personal care formulation according to claim 12 wherein the keratin protein fraction is from the high sulfur protein family.

16. A personal care formulation according to claim 14 wherein the cysteine content of the keratin protein is around 4%.

17. A personal care formulation according to claim 15 wherein the cysteine content of the keratin protein is greater than 10%.

18. A personal care formulation according to claim 12 wherein the keratin protein fraction is from the high glycine-tyrosine protein family.

19. An additive comprising an s-sulfonated keratin protein fraction, when used in a personal care formulation.

20. An additive according to claim 19 wherein the protein fraction is intact.

21. An additive according to claim 19 wherein the protein fraction is hydrolysed.

22. An additive according to any one of claims 19 to 21 wherein the protein fraction is from the intermediate filament protein family.

23. An additive according to claim 19 wherein the protein fraction is from the high sulfur protein family.

24. An additive according to claim 22 wherein the cysteine content of the protein is around 4%.

25. An additive according to claim 23 wherein the cysteine content of the protein is greater than 10%.

26. An additive according to claim 19 wherein the protein fraction is from the high glycine-tyrosine protein family.

27. An additive for a personal care formulation that contains from 0.001% to 50% of an s-sulfonated keratin protein fraction.

28. An additive according to claim 27 containing from 0.001% to 10% of an s-sulfonated keratin protein fraction.

29. An additive according to claim 29 containing from 0.001% to 1% of an s-sulfonated keratin protein fraction.

30. An additive according to claim 27 wherein the keratin protein fraction is intact.

31. An additive according to claim 27 wherein the keratin protein fraction is hydrolysed.

32. An additive according to claim 30 wherein the keratin protein fraction is from the intermediate filament protein family.

33. An additive according to claim 30 wherein the keratin protein fraction is from the high sulphur protein family.

34. An additive according to claim 32 wherein the cysteine content of the keratin protein is around 4%.

35. An additive according to claim 33 wherein the cysteine content of the keratin protein is greater than 10%.

36. An additive as claimed in claim 27 wherein the keratin protein fraction is from the high glycine-tyrosine protein family.

37. A method comprising using formulation as claimed in claim 1 for personal care.

38. A method of treating hair comprising the use of a formulation or additive according to claim 1 or 9.
